(19) United States
(12) Patent Application Publication
(10) Pub. No.: US 2010/0160960 A1
(43) Pub. Date: Jun. 24, 2010

(54) HYDROGEL TISSUE ADHESIVE HAVING INCREASED DEGRADATION TIME

(75) Inventors: Mark E. Wagman, Wilmington, DE (US); Kai Qi, Wilmington, DE (US); Keith Kunitsky, West Grove, PA (US); Hing Wan Yim, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/614,659

(22) Filed: Nov. 9, 2009

Related U.S. Application Data
(60) Provisional application No. 61/139,239, filed on Dec. 19, 2008.

(51) Int. Cl. A61B 17/03; C08K 5/05; C08L 5/00

(52) U.S. Cl. 606/214; 524/386; 524/27

(57) ABSTRACT

A hydrogel tissue adhesive having increased degradation time is described. The hydrogel tissue adhesive is formed by reacting an oxidized polysaccharide with a water-dispersible, multi-arm amine in the presence of a polyol additive, which retards the degradation of the hydrogel. The hydrogel may be useful as a tissue adhesive or sealant for medical applications, including but not limited to, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; as a plug to seal a fistula or the punctum; adhesion prevention to prevent undesired tissue to tissue adhesions resulting from trauma or surgery; and as a hemostat sealant.

### CROSS-REFERENCE TO RELATED APPLICATION

This application claims priority under 35 U.S.C. §119 from U.S. Provisional Application Ser. No. 61/139,239, filed Dec. 19, 2008.

### FIELD OF THE INVENTION

The invention relates to the field of medical adhesives. More specifically, the invention relates to a hydrogel tissue adhesive formed by reacting an oxidized polysaccharide with a water-dispersible, multi-arm amine wherein the hydrogel comprises a polyol additive which retards the degradation of the hydrogel.

### BACKGROUND OF THE INVENTION

Tissue adhesives have many potential medical applications, including wound closure, supplementing or replacing sutures or staples in internal surgical procedures, adhesion of synthetic onlays or inlays to the cornea, drug delivery devices, and as anti-adhesion barriers to prevent post-surgical adhesions. Conventional tissue adhesives are generally not suitable for a wide range of adhesive applications. For example, cyanoacrylate-based adhesives have been used for topical wound closure, but the release of toxic degradation products limits their use for internal applications. Fibrin-based adhesives are slow curing, have poor mechanical strength, and pose a risk of viral infection. Additionally, fibrin-based adhesives do not bond covalently to the underlying tissue.

Several types of hydrogel tissue adhesives have been developed, which have improved adhesive and cohesive properties and are nontoxic (see for example Sehl et al., U.S. Patent Application Publication No. 2003/0119985, and Goldmann, U.S. Patent Application Publication No. 2005/0002893). These hydrogels are generally formed by reacting a component having nucleophilic groups with a component having electrophilic groups, which are capable of reacting with the nucleophilic groups of the first component, to form a crosslinked network via covalent bonding. However, these hydrogels typically swell or dissolve away too quickly, or lack sufficient adhesion or mechanical strength, thereby decreasing their effectiveness as surgical adhesives. If the gelation time is too fast, the application device will readily clog.

Kodokian et al. (copending and commonly owned U.S. Patent Application Publication No. 2006/0078536) describe hydrogel tissue adhesives formed by reacting an oxidized polysaccharide with a water-dispersible, multi-arm polyether amine. These adhesives provide improved adhesion and cohesion properties, crosslink readily at body temperature, maintain dimensional stability initially, do not degrade rapidly, and are nontoxic to cells and non-inflammatory to tissue. For certain applications, for example, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; and as a plug to seal a fistula or the punctum, more slowly degrading hydrogel tissue adhesives are needed. Additionally, the gelation time to form the hydrogel tissue adhesive described by Kodokian is quite rapid, typically less than 10 seconds. Generally, adjustments to the oxidized polysaccharide/water-dispersible, multi-arm polyether amine formulation either slows gelation while decreasing degradation time, or speeds gelation while increasing degradation time. Therefore, it is difficult to increase the degradation time of the hydrogel without an undesired decrease in the gelation time.

Figuly et al. (copending and commonly owned U.S. patent application Ser. No. 12/145,737) describe a method for extending the gelation time of an oxidized polysaccharide to react with a water-dispersible, multi-arm amine to form a hydrogel. The method also decreases the degradation time of the hydrogel. The method utilizes a chemical additive that reacts with the functional groups of the oxidized polysaccharide or the water-dispersible, multi-arm amine, thereby reducing the number of groups available for crosslinking.

Therefore, the need exists for a hydrogel tissue adhesive that has the desirable properties of the tissue adhesive described by Kodokian et al., supra, but has a longer degradation time.

### SUMMARY OF THE INVENTION

The present invention addresses the above need by providing a hydrogel tissue adhesive formed by reacting an oxidized polysaccharide with a water-dispersible, multi-arm amine wherein the hydrogel comprises a polyol additive which retards the degradation of the hydrogel.

Accordingly, in one embodiment the invention provides a kit comprising:
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
   provided that:
      (i) the water-dispersible polyol does not induce gelation when mixed in an aqueous medium with either (a) alone or (b) alone; and
      (ii) if said water-dispersible polyol is a polysaccharide, then said polysaccharide has a weight-average molecular weight greater than 5,000 to about 2,000,000 Daltons.

In another embodiment, the invention provides a dried hydrogel formed by a process comprising the steps of:
a) combining in a solvent (i) at least one oxidized polysaccharide containing aldehyde groups, said oxidized polysaccharide having a weight-average molecular weight of about 1,000 to about 1,000,000 Daltons and having an equivalent weight per aldehyde group of about 65 to about 1500 Daltons with (ii) at least one water-dispersible, multi-arm polyether amine having at least three arms terminated by at least one primary amine group, said multi-arm polyether amine having a number-average molecular weight of about 450 to about 200,000 Daltons, and (iii) an effective amount of at least one water-dispersible polyol having a molecular weight greater than 2,000 to about 2,000,000 Daltons, said polyol containing two or more hydroxyl groups per molecule, to form a hydrogel, and
b) treating said hydrogel to remove at least a portion of said solvent to form the dried hydrogel;
provided that:
   (A) the water-dispersible polyol does not induce gelation when mixed in an aqueous medium with either (i) alone or (ii) alone; and
   (B) if said water-dispersible polyol is a polysaccharide, then said polysaccharide has a weight-average molecular weight greater than 5,000 to about 2,000,000 Daltons.

In another embodiment, the invention provides a composition comprising the reaction product of:
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
   provided that:
      (i) the water-dispersible polyol does not induce gelation when mixed in an aqueous medium with either (a) alone or (b) alone; and
      (ii) if said water-dispersible polyol is a polysaccharide, then said polysaccharide has a weight-average molecular weight greater than 5,000 to about 2,000,000 Daltons.

In another embodiment, the invention provides a method for applying a coating to an anatomical site on tissue of a living organism comprising:

applying to the site
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
   provided that:
      (i) the water-dispersible polyol does not induce gelation when mixed in an aqueous medium with either (a) alone or (b) alone; and
      (ii) if said water-dispersible polyol is a polysaccharide, then said polysaccharide has a weight-average molecular weight greater than 5,000 to about 2,000,000 Daltons;
wherein (a), (b), and (c) are applied to the site in any order, or (a), (b), and (c) are premixed and the resulting mixture is applied to the site before the mixture completely cures.

In another embodiment, the invention provides a method for bonding at least two anatomical sites together comprising:

applying to at least one of the at least two anatomical sites:
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
   provided that:
      (i) the water-dispersible polyol does not induce gelation when mixed in an aqueous medium with either (a) alone or (b) alone; and
      (ii) if said water-dispersible polyol is a polysaccharide, then said polysaccharide has a weight-average molecular weight greater than 5,000 to about 2,000,000 Daltons;
or premixing (a), (b) and (c) and applying the resulting mixture to at least one site before the resulting mixture completely cures; and contacting the at least two anatomical sites together.

In another embodiment, the invention provides a method for increasing degradation time of a hydrogel formed from at least one oxidized polysaccharide (component A) and at least one water-dispersible, multi-arm amine (component B), said at least one oxidized polysaccharide containing aldehyde groups, having a weight-average molecular weight of about 1,000 to about 1,000,000 Daltons and an equivalent weight per aldehyde group of about 65 to about 1500 Daltons, and said at least one water-dispersible, multi-arm amine having at least three arms terminated by at least one primary amine group, and a number-average molecular weight of about 450 to about 200,000 Daltons; said method comprising:

contacting component A and component B in the presence of an aqueous medium and at least one water-dispersible polyol having a weight-average molecular weight greater than 2,000 to about 2,000,000 Daltons, said polyol containing two or more hydroxyl groups per molecule to form a mixture that forms a resulting hydrogel, wherein, in said method, the water-dispersible polyol is used in an amount sufficient to increase the degradation time of the resulting hydrogel under predetermined conditions by at least about 10% compared to that of the hydrogel formed under said conditions, but in the absence of said water-dispersible polyol;

provided that:
(i) the water-dispersible polyol does not induce gelation when mixed in an aqueous medium with either component A alone or component B alone; and
(ii) if said water-dispersible polyol is a polysaccharide, then said polysaccharide has a weight-average molecular weight greater than 5,000 to about 2,000,000 Daltons.

### DETAILED DESCRIPTION

As used above and throughout the description of the invention, the following terms, unless otherwise indicated, shall be defined as follows:

The term “oxidized polysaccharide” refers to a polysaccharide which has been reacted with an oxidizing agent to introduce aldehyde groups into the molecule.

The term “equivalent weight per aldehyde group” refers to the molecular weight of the oxidized polysaccharide divided by the number of aldehyde groups introduced in the molecule.

The term “water-dispersible, multi-arm amine” refers to a polymer having three or more polymer chains (“arms”), which may be linear or branched, emanating from a central structure, which may be a single atom, a core molecule, or a polymer backbone, wherein at least three branches (“arms”) are terminated by at least one primary amine group. The water-dispersible, multi-arm amine is water soluble or is able to be dispersed in water to form a colloidal suspension capable of reacting with a second reactant in aqueous solution or dispersion.

The term “water-dispersible, multi-arm polyether amine” refers to a branched polyether, wherein at least three of the branches (“arms”) are terminated by at least one primary amine group, which is water soluble or able to be dispersed in water to form a colloidal dispersion capable of reacting with a second reactant in aqueous solution or dispersion.

The term “polyether” refers to a polymer having the repeat unit [—O—R]—, wherein R is a hydrocarbyl group having 2 to 5 carbon atoms. The polyether may also be a random or block copolymer comprising different repeat units.

The term “branched polyether” refers to a polyether having one or more branch points (“arms”), including star, dendritic, comb, and hyperbranched polyethers.

The term “dendritic polyether” refers to a highly branched polyether having a tree-like structure.

The term “comb polyether” refers to a polyether having a main chain with multiple trifunctional branch points from each of which a linear arm emanates.

The term “star polyether” refers to a polyether having a central branch point, which may be a single atom or a chemical group, from which linear arms emanate.

The term “hyperbranched polyether” refers to a highly branched polyether having fewer branches and less regular branching than a dendritic polyether.

The term “water-dispersible polyol” refers to a chemical compound having two or more OH groups, which is water soluble or able to be dispersed in water to form a colloidal dispersion.

The term “primary amine” refers to a neutral amino group having two free hydrogens. The amino group may be bound to a primary, secondary or tertiary carbon.

The term “multi-functional amine” refers to a chemical compound comprising at least two functional groups, at least one of which is a primary amine group.

The term “crosslink” refers to a bond or chain of atoms attached between and linking two different polymer chains.

The term “crosslink density” is herein defined as the reciprocal of the average number of chain atoms between crosslink connection sites.

The term “% by weight”, also referred to herein as “wt %” refers to the weight percent relative to the total weight of the solution or dispersion, unless otherwise specified.

The term “anatomical site” refers to any external or internal part of the body of humans or animals.

The term “tissue” refers to any biological tissue, both living and dead, in humans or animals.

The term “hydrogel” refers to a water-swellable polymeric matrix, consisting of a three-dimensional network of macromolecules held together by covalent or non-covalent crosslinks, that can absorb a substantial amount of water to form an elastic gel.

The term “PEG” as used herein refers to poly(ethylene glycol).

The term “M_{w}” as used herein refers to the weight-average molecular weight.

The term “Mₙ” as used herein refers to the number-average molecular weight.

The term “medical application” refers to medical applications as related to humans and animals.

The meaning of abbreviations used is as follows: “min” means minute(s), “h” means hour(s), “sec” means second(s), “d” means day(s), “mL” means milliliter(s), “L” means liter(s), “μL” means microliter(s), “cm” means centimeter(s), “mm” means millimeter(s), “μm” means micrometer(s), “mol” means mole(s), “mmol” means millimole(s), “g” means gram(s), “mg” means milligram(s), “wt %” means percent by weight, “mol %” means mole percent, “Vol” means volume, “v/v” means volume per volume, “Da” means Daltons, “kDa” means kiloDaltons, the designation “10K” means that a polymer molecule possesses a number-average molecular weight of 10 kiloDaltons, “M” means molarity, “kPa” means kilopascals. “¹H NMR” means proton nuclear magnetic resonance spectroscopy, “ppm” means parts per million, “PBS” means phosphate-buffered saline.

Disclosed herein is a hydrogel tissue adhesive formed by reacting an oxidized polysaccharide with a water-dispersible, multi-arm amine wherein the hydrogel comprises a polyol additive which retards the degradation of the hydrogel. The hydrogel may be useful as a tissue adhesive or sealant for medical applications that require a long degradation time, including but not limited to, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; and as a plug to seal a fistula or the punctum. The hydrogel may also be useful for medical applications that require a combination of resistance to applicator clogging and adequate degradation time, such as anti-adhesion barriers.

### Oxidized Polysaccharides:

Oxidized polysaccharides useful in the invention include, but are not limited to, oxidized derivatives of dextran, carboxymethyldextran, starch, agar, cellulose, hydroxyethylcellulose, carboxymethylcellulose, pullulan, inulun, levan, and hyaluronic acid. The starting polysaccharides are available commercially from sources such as Sigma Chemical Co. (St. Louis, Mo.). Typically, polysaccharides are a heterogeneous mixture having a distribution of different molecular weights, and are characterized by an average molecular weight, for example, the weight-average molecular weight (M_{w}), or the number average molecular weight (Mₙ), as is known in the art. Suitable oxidized polysaccharides have a weight-average molecular weight of about 1,000 to about 1,000,000 Daltons, more particularly about 3,000 to about 250,000 Daltons, more particularly about 5,000 to about 100,000 Daltons, and more particularly about 10,000 to about 60,000 Daltons. In one embodiment, the oxidized polysaccharide is oxidized dextran, also referred to herein as dextran aldehyde.

Oxidized polysaccharides may be prepared by oxidizing a polysaccharide to introduce aldehyde groups using any suitable oxidizing agent, including but not limited to, periodates, hypochlorites, ozone, peroxides, hydroperoxides, persulfates, and percarbonates. For example, the polysaccharide may be oxidized by reaction with sodium periodate as described by Mo et al. (J. Biomater. Sci. Polymer Edn. 11:341-351, 2000). The polysaccharide may be reacted with different amounts of periodate to give polysaccharides with different degrees of oxidation and therefore, different amounts of aldehyde groups. Additionally, the oxidized polysaccharide may be prepared using the method described by Cohen et al. (copending and commonly owned Patent Application Publication No. WO 2008/133847). That method of making an oxidized polysaccharide comprises a combination of precipitation and separation steps to purify the oxidized polysaccharide formed by oxidation of the polysaccharide with periodate, as described in detail in the Examples herein below, and provides an oxidized polysaccharide with very low levels of iodine-containing species. The degree of oxidation, also referred to herein as the oxidation conversion, of the oxidized polysaccharide may be determined using methods known in the art. For example, the degree of oxidation of the oxidized polysaccharide may be determined using the method described by Hofreiter et al. (Anal Chem. 27:1930-1931, 1955). In that method, the amount of alkali consumed per mole of dialdehyde in the oxidized polysaccharide, under specific reaction conditions, is determined by a pH titration. Alternatively, the degree of oxidation of the oxidized polysaccharide may be determined using nuclear magnetic resonance (NMR) spectroscopy, as described in detail in the Examples herein below. In one embodiment, the equivalent weight per aldehyde group of the oxidized polysaccharide is from about 65 to about 1500 Daltons, more particularly from about 90 to about 1500 Daltons.

### Water-Dispersible, Multi-Arm Amines:

Suitable water dispersible, multi-arm amines include, but are not limited to, water dispersible multi-arm polyether amines, amino-terminated dendritic polyamidoamines, and multi-arm branched end amines. Typically, the multi-arm amines have a number-average molecular weight of about 450 to about 200,000 Daltons, more particularly from about 2,000 to about 40,000 Daltons.

In one embodiment, the water dispersible, multi-arm amine is a multi-arm polyether amine, which is a water-dispersible polyether having the repeat unit [—O—R]—, wherein R is a hydrocarbylene group having 2 to 5 carbon atoms. The term “hydrocarbylene group” refers to a divalent group formed by removing two hydrogen atoms, one from each of two different carbon atoms, from a hydrocarbon. Suitable multi-arm polyether amines include, but are not limited to, amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, and polyoxyalkylene triamines, sold under the trade name Jeffamine® triamines, by Huntsman LLC. (Houston, Tex.). Examples of star polyethylene oxide amines, include, but are not limited to, various multi-arm polyethylene glycol amines, and star polyethylene glycols having 3, 4, 6, or 8 arms terminated with primary amines (referred to herein as 3, 4, 6, or 8-arm star PEG amines, respectively). Examples of suitable Jeffaminetriamines include, but are not limited to, Jeffamine® T-403 (CAS No. 39423-51-3), Jeffamine® T-3000 (CAS No. 64852-22-8), and Jeffamine® T-5000 (CAS No. 64852-22-8). In one embodiment, the water-dispersible multi-arm polyether amine is an eight-arm polyethylene glycol having eight arms terminated by a primary amine group and having a number-average molecular weight of about 10,000 Daltons.

The multi-arm polyether amines are either available commercially, as noted above, or may be prepared using methods known in the art. For example, multi-arm polyethylene glycols, wherein at least three of the arms are terminated by a primary amine group, may be prepared by putting amine ends on multi-arm polyethylene glycols (e.g., 3, 4, 6, and 8-arm star polyethylene glycols, available from companies such as Nektar Transforming Therapeutics; SunBio, Inc., Anyang City, South Korea; NOF Corp., Tokyo, Japan; or JenKem Technology USA, Allen, Tex.) using the method described by Buckmann et al. (Makromol. Chem. 182:1379-1384, 1981). In that method, the multi-arm polyethylene glycol is reacted with thionyl bromide to convert the hydroxyl groups to bromines, which are then converted to amines by reaction with ammonia at 100° C. The method is broadly applicable to the preparation of other multi-arm polyether amines. Additionally, multi-arm polyether amines may be prepared from multi-arm polyols using the method described by Chenault (copending and commonly owned U.S. Patent Application Publication No. 2007/0249870). In that method, the multi-arm polyether is reacted with thionyl chloride to convert the hydroxyl groups to chlorine groups, which are then converted to amines by reaction with aqueous or anhydrous ammonia. Other methods that may be used for preparing multi-arm polyether amines are described by Merrill et al. in U.S. Pat. No. 5,830,986, and by Chang et al. in WO 97/30103.

The water-dispersible, multi-arm amine may also be an amino-terminated dendritic polyamidoamine, sold under the trade name Starburst® Dendrimers (available from Sigma-Aldrich, St Louis, Mo.).

The water-dispersible, multi-arm amine may also be a multi-arm branched end amine, as described by Arthur (copending and commonly owned Patent Application Publication No. WO 2008/066787). The multi-arm branched end amines are branched polymers having two or three amine groups at the end of the polymer arms. The multiplicity of functional groups increases the statistical probability of reaction at a given chain end and allows more efficient incorporation of the branched molecules into a polymer network. The starting materials used to prepare the branched end amines may be branched polymers such as multi-arm polyether polyols including, but not limited to, comb and star polyether polyols. The branched end amines can be prepared by attaching multiple amine groups to the ends of the polymer by reaction with the hydroxyl groups using methods well known in the art. For example, a branched end amine having two amine functional groups on each end of the polymer arms can prepared by reacting the starting material, as listed above, with thionyl chloride in a suitable solvent such as toluene to give the chloride derivative, which is subsequently reacted with tris(2-aminoethyl)amine to give the branched end reactant having two primary amine groups at the end of the polymer arms.

It should be recognized that the water-dispersible, multi-arm amines are generally a somewhat heterogeneous mixture having a distribution of arm lengths and in some cases, a distribution of species with different numbers of arms. When a multi-arm amine has a distribution of species having different numbers of arms, it can be referred to based on the average number of arms in the distribution. For example, in one embodiment the multi-arm amine is an 8-arm star PEG amine, which comprises a mixture of multi-arm star PEG amines, some having less than and some having more than 8 arms; however, the multi-arm star PEG amines in the mixture have an average of 8 arms. Therefore, the terms “8-arm”, “6-arm”, “4-arm” and “3-arm” as used herein to refer to multi-arm amines, should be construed as referring to a heterogeneous mixture having a distribution of arm lengths and in some cases, a distribution of species with different numbers of arms, in which case the number of arms recited refers to the average number of arms in the mixture.

In one embodiment, the oxidized polysaccharide is oxidized dextran and the water-dispersible, multi-arm amine is a multi-arm polyethylene glycol amine.

### Water-Dispersible Polyols:

The water-dispersible polyol additive serves to extend the degradation time of the hydrogel resulting from the reaction of the oxidized polysaccharide and the water-dispersible, multi-arm amine, and may or may not decrease the rate of gelation to form the hydrogel, depending on the particular additive and concentration used. Suitable water-dispersible polyols have a weight-average molecular weight greater than 2,000 Daltons to about 2,000,000 Daltons, more particularly about 4,000 Daltons to about 2,000,000 Daltons, and more particularly, about 4,000 Daltons to about 35,000 Daltons, and contain two or more hydroxyl groups per molecule. Additionally, the water-dispersible polyol does not induce gelation when mixed in an aqueous medium with either an oxidized polysaccharide alone or a multi-arm amine alone. For example, if the water-dispersible polyol does not contain other functional groups capable of reacting to form a covalent bond with either primary amine groups or aldehyde groups, it will not induce gelation when mixed in an aqueous medium with either an oxidized polysaccharide alone or a multi-arm amine alone. If the water-dispersible polyol does contain other functional groups capable of reacting to form a covalent bond with either primary amine groups or aldehyde groups, then the density of the functional groups is low so that gelation is not induced when the water-dispersible polyol is mixed in an aqueous medium with either an oxidized polysaccharide alone or a multi-arm amine alone.

Examples of useful water-dispersible polyols include, but are not limited to, linear or branched poly(ethylene glycol), block or random copolymers of ethylene glycol and propylene glycol, poly(vinyl alcohol), polysaccharides, such as dextran, and combinations thereof. If the water-dispersible polyol is a polysaccharide, then the polysaccharide has a weight-average molecular weight greater than 5,000 Daltons to about 2,000,000 Daltons, more particularly about 10,000 Daltons to about 2,000,000 Daltons.

In one embodiment, the water-dispersible polyol is a block copolymer of ethylene oxide and propylene oxide. In another embodiment, the water-dispersible polyol is a random copolymer of ethylene glycol and propylene glycol. In another embodiment, the water-dispersible polyol is a linear poly(ethylene glycol). In another embodiment, the water-dispersible polyol is an 8-arm poly(ethylene glycol). In another embodiment, the water-dispersible polyol is poly(vinyl alcohol). In another embodiment, the water-dispersible polyol is dextran.

### Methods of Using the Hydrogel Tissue Adhesive

The hydrogel tissue adhesive disclosed herein may be used in various forms. In one embodiment, the oxidized polysaccharide containing aldehyde groups, the water-dispersible, multi-arm amine, and the water-dispersible polyol are used in the form of aqueous solutions or dispersions. Dispersion, as used herein, refers to a colloidal suspension capable of reacting with a second reactant in an aqueous medium. To prepare an aqueous solution or dispersion comprising an oxidized polysaccharide (referred to herein as the “first aqueous solution or dispersion”), at least one oxidized polysaccharide is added to water to give a concentration of about 5% to about 40%, more particularly from about 5% to about 30%, and more particularly from about 10% to about 30% by weight relative to the total weight of the solution or dispersion. Additionally, a mixture of at least two different oxidized polysaccharides having different weight-average molecular weights, different degrees of oxidation, or both different weight-average molecular weights and different degrees of oxidation may be used. Where a mixture of oxidized polysaccharides is used, the total concentration of the oxidized polysaccharides is about 5% to about 40% by weight, more particularly from about 5% to about 30%, and more particularly from about 10% to about 30% by weight relative to the total weight of the solution or dispersion.

Similarly, to prepare an aqueous solution or dispersion comprising a water-dispersible, multi-arm amine (referred to herein as the “second aqueous solution or dispersion”), at least one water-dispersible, multi-arm amine is added to water to give a concentration of about 5% to about 70% by weight, in addition from about 20% to about 50% by weight relative to the total weight of the solution or dispersion. The optimal concentration to be used depends on the intended application and on the concentration of the oxidized polysaccharide used in the first aqueous solution or dispersion. Additionally, a mixture of different water-dispersible, multi-arm amines having different number-average molecular weights, different numbers of arms, or both different number-average molecular weights and different numbers of arms may be used. Where a mixture of water-dispersible, multi-arm amines is used, the total concentration of the multi-arm amines is about 5% to about 70% by weight, more particularly from about 20% to about 50% by weight relative to the total weight of the solution or dispersion.

An effective amount of at least one water-dispersible polyol is added to at least one of the following solutions or dispersions: the aqueous solution or dispersion comprising the oxidized polysaccharide (i.e., the first aqueous solution or dispersion), the aqueous solution or dispersion comprising the water-dispersible, multi-arm amine (i.e., the second aqueous solution or dispersion), or a third aqueous solution or dispersion. The water-dispersible polyol has the greatest effect on the degradation time of the hydrogel when it is added to the aqueous solution or dispersion comprising the oxidized polysaccharide. The solutions or dispersions containing the water-dispersible polyol may be prepared in any number of ways. For example, the water-dispersible polyol may be added to the first aqueous solution or dispersion containing the oxidized polysaccharide, prepared as described above, or the second aqueous solution or dispersion containing the multi-arm amine, prepared as described above. Alternatively, the water-dispersible polyol may first be dissolved in water and the oxidized polysaccharide or multi-arm amine may be subsequently added. Additionally an aqueous solution or dispersion containing the water-dispersible polyol may be combined with either the first or second aqueous solutions or dispersions. An effective amount of the water-dispersible polyol is an amount sufficient to provide the desired degradation time for the hydrogel. In general, the larger the amount of the water-dispersible polyol used, the greater is the effect on extending the degradation time of the hydrogel. The amount of the water-dispersible polyol to be used to achieve the desired degradation time can be determined by one skilled in the art using routine experimentation. In one embodiment, an effective amount of the water-dispersible polyol is about 1 to about 30 wt %, more particularly, about 5 to about 20 wt %.

In one embodiment, the amount of water-dispersible polyol used is sufficient to provide an increase in degradation time, under predetermined conditions, of at least about 10% compared to that of the hydrogel formed under the same conditions, but in the absence of the water-dispersible polyol. For any set of predetermined conditions, the degradation time of the resulting hydrogel can be determined using methods known in the art. For example, after the hydrogel is formed, it can be incubated in an aqueous medium with shaking at a specified temperature and agitation speed and the time required for the gel to dissolve can be measured, as described in the Examples herein below.

For use on living tissue, it is preferred that the first aqueous solution or dispersion and the second aqueous solution or dispersion be sterilized to prevent infection. Any suitable sterilization method known in the art that does not adversely affect the ability of the components to react to form an effective hydrogel may be used, including, but not limited to, electron beam irradiation, gamma irradiation, ethylene oxide sterilization, or ultra-filtration through a 0.2 μm pore membrane. If the water-dispersible polyol is contained in a third aqueous solution or dispersion, that solution or dispersion can also be sterilized using the methods listed above.

The first aqueous solution or dispersion, the second aqueous solution or dispersion, and/or the third aqueous solution or dispersion (if used) may further comprise various additives depending on the intended application. Preferably, the additive does not interfere with effective gelation to form a hydrogel. The amount of the additive used depends on the particular application and may be readily determined by one skilled in the art using routine experimentation. For example, the first aqueous solution or dispersion, the second aqueous solution or dispersion, and/or the third aqueous solution or dispersion may comprise at least one additive selected from pH modifiers, antimicrobials, colorants, surfactants, pharmaceutical drugs and therapeutic agents.

The first aqueous solution or dispersion, the second aqueous solution or dispersion, and/or the third aqueous solution or dispersion may optionally include at least one pH modifier to adjust the pH of the solution(s) or dispersion(s). Suitable pH modifiers are well known in the art. The pH modifier may be an acidic or basic compound. Examples of acidic pH modifiers include, but are not limited to, carboxylic acids, inorganic acids, and sulfonic acids. Examples of basic pH modifiers include, but are not limited to, hydroxides, alkoxides, nitrogen-containing compounds other than primary and secondary amines, and basic carbonates and phosphates.

The first aqueous solution or dispersion, the second aqueous solution or dispersion, and/or the third aqueous solution or dispersion may optionally include at least one antimicrobial agent. Suitable antimicrobial preservatives are well known in the art. Examples of suitable antimicrobials include, but are not limited to, alkyl parabens, such as methylparaben, ethylparaben, propylparaben, and butylparaben; triclosan; chlorhexidine; cresol; chlorocresol; hydroquinone; sodium benzoate; and potassium benzoate.

The first aqueous solution or dispersion, the second aqueous solution or dispersion, and/or the third aqueous solution or dispersion may optionally include at least one colorant to enhance the visibility of the solution(s) or dispersion(s). Suitable colorants include dyes, pigments, and natural coloring agents. Examples of suitable colorants include, but are not limited to, FD&C and D&C colorants, such as FD&C Violet No. 2, FD&C Blue No. 1, D&C Green No. 6, D&C Green No. 5, D&C Violet No. 2; and natural colorants such as beetroot red, canthaxanthin, chlorophyll, eosin, saffron, and carmine.

The first aqueous solution or dispersion, the second aqueous solution or dispersion, and/or the third aqueous solution or dispersion may optionally include at least one surfactant. Surfactant, as used herein, refers to a compound that lowers the surface tension of water. The surfactant may be an ionic surfactant, such as sodium lauryl sulfate, or a neutral surfactant, such as polyoxyethylene ethers, polyoxyethylene esters, and polyoxyethylene sorbitan.

Additionally, the first aqueous solution or dispersion, the second aqueous solution or dispersion, and/or the third aqueous solution or dispersion may optionally include at least one pharmaceutical drug or therapeutic agent. Suitable drugs and therapeutic agents are well known in the art (for example see the United States Pharmacopeia (USP), Physician's Desk Reference (Thomson Publishing), The Merck Manual of Diagnosis and Therapy 18th ed., Mark H. Beers and Robert Berkow (eds.), Merck Publishing Group, 2006; or, in the case of animals, The Merck Veterinary Manual, 9th ed., Kahn, C. A. (ed.), Merck Publishing Group, 2005). Nonlimiting examples include, but are not limited to, anti-inflammatory agents, for example, glucocorticoids such as prednisone, dexamethasone, budesonide; non-steroidal anti-inflammatory agents such as indomethacin, salicylic acid acetate, ibuprofen, sulindac, piroxicam, and naproxen; fibrinolytic agents such as a tissue plasminogen activator and streptokinase; anti-coagulants such as heparin, hirudin, ancrod, dicumarol, sincumar, iloprost, L-arginine, dipyramidole and other platelet function inhibitors; antibodies; nucleic acids; peptides; hormones; growth factors; cytokines; chemokines; clotting factors; endogenous clotting inhibitors; antibacterial agents; antiviral agents; antifungal agents; anti-cancer agents; cell adhesion inhibitors; healing promoters; vaccines; thrombogenic agents, such as thrombin, fibrinogen, homocysteine, and estramustine; radio-opaque compounds, such as barium sulfate and gold particles and radiolabels.

Additionally, the second aqueous solution or dispersion comprising the multi-arm amine may optionally comprise at least one other multi-functional amine having one or more primary amine groups to provide other beneficial properties, such as hydrophobicity or modified crosslink density. The multi-functional amine is capable of inducing gelation when mixed with an oxidized polysaccharide in an aqueous solution or dispersion. The multi-functional amine may be a second water dispersible, multi-arm amine, such as those described above, or another type of multi-functional amine, including, but not limited to, linear and branched diamines, such as diaminoalkanes, polyaminoalkanes, and spermine; branched polyamines, such as polyethylenimine; cyclic diamines, such as N,N′-bis(3-aminopropyl)piperazine, 5-amino-1,3,3-trimethylcyclohexanemethylamine, 1,3-bis(aminomethyl)cyclohexane, 1,4-diaminocyclohexane, and p-xylylenediamine; aminoalkyltrialkoxysilanes, such as 3-aminopropyltrimethoxysilane and 3-aminopropyltriethoxysilane; aminoalkyldialkoxyalkylsilanes, such as 3-aminopropyldiethoxymethylsilane, dihydrazides, such as adipic dihydrazide; linear polymeric diamines, such as linear polyethylenimine, α,ω-amino-terminated polyethers, α,ω-bis(3-aminopropyl)polybutanediol, β,ω-1-amino-terminated polyethers (linear Jeffamines®); comb polyamines, such as chitosan, polyallylamine, and polylysine, and di- and polyhydrazides, such as bis(carboxyhydrazido)polyethers and poly(carboxyhydrazido) star polyethers. Many of these compounds are commercially available from companies such as Sigma-Aldrich and Huntsman LLC. Typically, if present, the multi-functional amine is used at a concentration of about 5% by weight to about 1000% by weight relative to the weight of the multi-arm amine in the aqueous solution or dispersion.

The first aqueous solution or dispersion and the second aqueous solution or dispersion may be used to apply a coating to an anatomical site on tissue of a living organism. The two aqueous solutions or dispersions may be applied to the site in any number of ways. Once both solutions or dispersions are combined on a site, they crosslink to form a hydrogel which provides a coating on the site.

In one embodiment wherein the water-dispersible polyol is contained in at least one of the first aqueous solution or dispersion or the second aqueous solution or dispersion, the two aqueous solutions or dispersions are applied to the site sequentially using any suitable means including, but not limited to, spraying, brushing with a cotton swab or brush, or extrusion using a pipette, or a syringe. The solutions or dispersions may be applied in any order. Then, the solutions or dispersions are mixed on the site using any suitable device, such as a cotton swab, a spatula, or the tip of the pipette or syringe.

In another embodiment, the two aqueous solutions or dispersions are mixed manually before application to the site. The resulting mixture is then applied to the site before it completely cures using a suitable applicator, as described above.

In another embodiment, the first aqueous solution or dispersion and the second aqueous solution or dispersion are applied to the site simultaneously where they mix to form a hydrogel. For example, the two aqueous solutions or dispersions may be contained in separate barrels of a double-barrel syringe. In this way the two aqueous solutions or dispersions are applied simultaneously to the site with the syringe. Suitable double-barrel syringe applicators are known in the art. For example, Redl describes several suitable applicators for use in the invention in U.S. Pat. No. 6,620,125, (particularly FIGS. 1, 5, and 6, which are described in Columns 4, line 10 through column 6, line 47). The two aqueous solutions or dispersions may also be applied to the site using a dual-lumen catheter, such as those available from Bistech, Inc. (Woburn, Mass.). Additionally, injection devices for introducing two liquid components endoscopically into the body simultaneously are known in the art and may be adapted for the delivery of the two aqueous solutions or dispersions disclosed herein (see for example, Linder et al., U.S. Pat. No. 5,322,510).

In another embodiment, the first aqueous solution or dispersion and the second aqueous solution or dispersion may be premixed and delivered to the site using a double barrel syringe containing a motionless mixer, such as that available from ConProtec, Inc. (Salem, N.H.) or Mixpac Systems AG (Rotkreuz, Switzerland). Alternatively, the mixing tip may be equipped with a spray head, such as that described by Cruise et al. in U.S. Pat. No. 6,458,147. Additionally, the mixture of the two aqueous solutions or dispersions from the double-barrel syringe may be applied to the site using a catheter or endoscope. Devices for mixing a two liquid component tissue adhesive and delivering the resulting mixture endoscopically are known in the art and may be adapted for the mixing and delivery of the two aqueous solutions or dispersions disclosed herein (see for example, Nielson, U.S. Pat. No. 6,723,067; and Redl et al., U.S. Pat. No. 4,631,055).

In another embodiment, the two aqueous solutions or dispersions may be applied to the site using a spray device, such as those described by Fukunaga et al. (U.S. Pat. No. 5,582,596), Delmotte et al. (U.S. Pat. No. 5,989,215) or Sawhney (U.S. Pat. No. 6,179,862).

In another embodiment, the two aqueous solutions or dispersions may be applied to the site using a minimally invasive surgical applicator, such as to those described by Sawhney (U.S. Pat. No. 7,347,850).

In another embodiment wherein the water-dispersible polyol is contained in a third aqueous solution or dispersion, the three aqueous solutions or dispersions may be applied to the anatomical site in any order using any of the methods described above. In this embodiment, the delivery device used may be modified to deliver the three aqueous solutions or dispersions. For example, the double-barrel syringe may be modified to have three barrels, one for each of the aqueous solutions or dispersions.

In another embodiment, the hydrogel tissue adhesive of the invention is used to bond at least two anatomical sites together. In one embodiment wherein the water-dispersible polyol is contained in at least one of the first aqueous solution or dispersion or the second aqueous solution or dispersion, the first aqueous solution or dispersion is applied to at least one anatomical site, and the second aqueous solution or dispersion is applied to at least one of either the same site or one other site using the methods described above. The two or more sites are contacted and held together manually or using some other means, such as a surgical clamp, for a time sufficient for the mixture to cure. Alternatively, a mixture of the two aqueous solutions or dispersions is applied to at least one of the anatomical sites to be bonded using methods described above. The two or more sites are contacted and held together manually or using some other means, such as a surgical clamp, for a time sufficient for the mixture to cure.

In another embodiment wherein the water-dispersible polyol is contained in a third aqueous solution or dispersion and used along with the first aqueous solution or dispersion and the second aqueous solution or dispersion to bond at least two anatomical sites together, each of the three aqueous solutions or dispersions may be applied to at least one anatomical site in any order. The aqueous solutions or dispersions may be applied to the same site or to different sites. Alternatively, the three aqueous solutions or dispersions may be premixed using any of the methods described above, and the resulting mixture applied to at least one of the anatomical sites to be bonded before the mixture completely cures. The two or more sites are then contacted and held together manually or using some other means, such as a surgical clamp, for a time sufficient for the mixture to cure.

In another embodiment, the oxidized polysaccharide, the water-dispersible, multi-arm amine, and the water-dispersible polyol may be used in the form of finely divided powders. The powders may be prepared using any suitable method. For example, the aqueous solutions or dispersions described above may be dried using heat, vacuum, a combination of heat and vacuum, or by lyophilization, to form powders. Optionally, the powders may be comminuted into finer particles using methods known in the art including, but not limited to, grinding, milling, or crushing with a mortar and pestle. The finely divided powders may be sterilized using the methods described above. The finely divided powders may be applied to an anatomical site on tissue of a living organism in a variety of ways. For example, the powders may be individually applied to the site in any order by sprinkling or spraying. Additionally, the powders may be premixed and the resulting mixture applied to the site by sprinkling or spraying. The powders may be hydrated on the site by the addition of an aqueous solution such as water or a suitable buffer (e.g., phosphate-buffered saline) or by the physiological fluids present at the site. The finely divided powders may also be used to bond two anatomical sites together as described above for the aqueous solutions or dispersions. Alternatively, the powders may be hydrated with water or a suitable aqueous solution prior to use to form the first and second aqueous solutions or dispersions, described above.

In another embodiment, the hydrogel tissue adhesive disclosed herein may be used in the form of a dried hydrogel. In this embodiment, a dried hydrogel is prepared by combining in a solvent at least one oxidized polysaccharide with at least one water-dispersible, multi-arm polyether amine and at least one water-dispersible polyol to form a hydrogel, and treating the hydrogel to remove at least a portion of the solvent to form the dried hydrogel. Suitable solvents include, but are not limited to, water, ethanol, isopropanol, tetrahydrofuran, hexanes, polyethylene glycol, and mixtures thereof. If two different solvents are used the two solvents are miscible with each other. In one embodiment, the solvent is water. The oxidized polysaccharide, the water-dispersible, multi-arm polyether amine, and the water-dispersible polyol may be combined in various ways. For example, the first aqueous solution or dispersion comprising the oxidized polysaccharide and the second aqueous solution or dispersion comprising the water-dispersible, multi-arm polyether amine, at least one of which comprises the water-dispersible polyol, may be prepared and mixed as described above to form the hydrogel. Alternatively, the first aqueous solution or dispersion comprising the oxidized polysaccharide, the second aqueous solution or dispersion comprising the water-dispersible, multi-arm polyether amine, and a third aqueous solution or dispersion comprising the water-dispersible polyol may be prepared and mixed as described above to form the hydrogel. The solutions or dispersions used to prepare the hydrogel may further comprise various additives depending on the intended application. Any of the additives described above may be used. The hydrogel is then treated to remove at least a portion of the solvent contained therein to form the dried hydrogel. Preferably, substantially all of the solvent is removed from the hydrogel. The solvent may be removed from the hydrogel using methods known in the art, for example, using heat, vacuum, a combination of heat and vacuum, or flowing a stream of dry air or a dry inert gas such as nitrogen over the hydrogel. The dried hydrogel may be sterilized using the methods described above. The dried hydrogel may be applied to an anatomical site in a number of ways, as described below. The dried hydrogel may be hydrated on the site by the addition of a suitable aqueous solution such as water or a buffer (e.g., phosphate-buffered saline) or by the physiological fluids present at the site.

In one embodiment, the dried hydrogel may be used in the form of a film. The dried hydrogel film may be formed by casting a mixture of the solutions or dispersions on a suitable substrate and treating the resulting hydrogel to form a dried hydrogel film. The dried hydrogel film may be applied directly to an anatomical site. Additionally, the dried hydrogel film may be used to bond two anatomical sites together.

In another embodiment, the dried hydrogel may be used in the form of finely divided particles. The dried hydrogel particles may be formed by comminuting the dried hydrogel using methods known in the art, including, but not limited to, grinding, milling, or crushing with a mortar and pestle. The dried hydrogel may be applied to an anatomical site in a variety of ways, such as sprinkling or spraying, and may also be used to bond two anatomical sites together.

### Method for Increasing Degradation Time:

In another embodiment, the invention provides a method for increasing the degradation time of a hydrogel formed from at least one oxidized polysaccharide (component A) and at least one water-dispersible, multi-arm amine (component B), said at least one oxidized polysaccharide containing aldehyde groups, having a weight-average molecular weight of about 1,000 to about 1,000,000 Daltons and an equivalent weight per aldehyde group of about 65 to about 1500 Daltons, and said at least one water-dispersible, multi-arm amine having at least three arms terminated by at least one primary amine group, and a number-average molecular weight of about 450 to about 200,000 Daltons; said method comprising:

contacting component A and component B in the presence of an aqueous medium and at least one water-dispersible polyol having a weight-average molecular weight greater than 2,000 to about 2,000,000 Daltons, said polyol containing two or more hydroxyl groups per molecule to form a mixture that forms a resulting hydrogel, wherein, in said method, the water-dispersible polyol is used in an amount sufficient to increase the degradation time of the resulting hydrogel under predetermined conditions by at least about 10% compared to that of the hydrogel formed under said conditions, but in the absence of said water-dispersible polyol;

provided that:
(i) the water-dispersible polyol does not induce gelation when mixed in an aqueous medium with either component A alone or component B alone; and
(ii) if said water-dispersible polyol is a polysaccharide, then said polysaccharide has a weight-average molecular weight greater than 5,000 to about 2,000,000 Daltons.

For any set of predetermined conditions, the degradation time of the resulting hydrogel can be determined using methods known in the art. For example, after the hydrogel is formed, it can be incubated in an aqueous medium with shaking at a specified temperature and agitation speed and the time required for the gel to dissolve can be measured, as described in the Examples herein below. The addition of the water dispersible polyol under the same predetermined conditions results in increasing the degradation time by at least about 10%.

### Kits

In one embodiment, the invention provides a kit comprising at least one oxidized polysaccharide containing aldehyde groups, at least one water-dispersible multi-arm amine having at least three arms terminated by at least one primary amine group, and an effective amount of at least one water-dispersible polyol, as described above.

In another embodiment, the kit comprises a first aqueous solution comprising at least one oxidized polysaccharide containing aldehyde groups, a second aqueous solution comprising at least one water-dispersible, multi-arm amine, and an effective amount of at least one water-dispersible polyol. The water-dispersible polyol is contained in at least one of the first aqueous solution or dispersion, the second aqueous solution or dispersion, or a third aqueous solution or dispersion, as described above. Each of the aqueous solutions or dispersions may be contained in any suitable vessel, such as a vial or a syringe barrel.

In another embodiment, the kit comprises at least one oxidized polysaccharide, at least one water-dispersible multi-arm amine, and at least one water-dispersible polyol in the form of finely divided powders, as described above. The powders may be contained in separate containers or they may be premixed and contained in a single container. The kit may also comprise an aqueous solution for hydrating the powders.

In another embodiment, the kit comprises a dried hydrogel as described above. The dried hydrogel may be in the form of a film, finely divided particles, or other dried forms. The kit may further comprise an aqueous solution for hydrating the dried hydrogel. The dried hydrogel particles may be contained in any suitable container.

### Medical Applications:

The hydrogel disclosed herein may be useful as a tissue adhesive or sealant for medical applications, including but not limited to, ophthalmic applications such as sealing wounds resulting from trauma such as corneal lacerations, or from surgical procedures such as vitrectomy procedures, cataract surgery, LASIK surgery, glaucoma surgery, and corneal transplants; neurosurgery applications, such as sealing the dura; and as a plug to seal a fistula or the punctum; adhesion prevention to prevent undesired tissue to tissue adhesions resulting from trauma or surgery; and as a hemostat sealant. In these applications, the oxidized polysaccharide, the water-dispersible multi-arm amine, and the water-dispersible polyol or the dried hydrogel may be applied to the desired anatomical site using the methods described above.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

A reference to “Aldrich” or a reference to “Sigma” means the said chemical or ingredient was obtained from Sigma-Aldrich, St. Louis, Mo.

### Reagents

The following reagents were used in the Examples:

Poloxamer 407—block copolymer of ethylene oxide (EO) and propylene oxide (PO) with average formula (EO)101-(P0)56-(EO)101, Mₙ=9,840 to 14,600 (Lutrol® F 127; BASF Corp., Florham Park, N.J.);

Poloxamer 188—block copolymer of ethylene oxide (EO) and propylene oxide (PO) with average formula (EO)79—(PO)28-(EO)79, Mₙ=7,680 to 9,510 (Lutrol® F 68, BASF Corp.);

Linear PEG 4.6 k—linear poly(ethylene glycol) di-alcohol, Mₙ=4,400 to 4,800 (Aldrich, cat. no. 373001);

Linear PEG 8k—linear poly(ethylene glycol) di-alcohol, Mₙ=7,000 to 9,000 (Aldrich, cat. no. 202452);

Linear PEG 14k—linear poly(ethylene glycol) di-alcohol, Mₙ=14,000 (Aldrich, cat. no. 637726);

P8-10-1-OH—8-arm poly(ethylene glycol) with each arm terminated by a hydroxyl group, (Mₙ=10000; NOF SunBright HGEO-10000 obtained from NOF America Corp., White Plains, N.Y.)

PVOH 88—poly(vinyl alcohol), 87-89% hydrolyzed, average M_{w}=13,000-23,000 (Aldrich, cat. no. 363170);

PVOH 98—poly(vinyl alcohol), 98% hydrolyzed, average M_{w}=13,000-23,000 (Aldrich, cat. no. 348406);

Poly(EG-ran-PG)—random copolymer of ethylene glycol and propylene glycol, Mₙ=12,000 (Aldrich, cat. no. 438200).

### Preparation of Dextran Aldehyde (D10-50):

Dextran aldehyde is made by oxidizing dextran in aqueous solution with sodium metaperiodate. An oxidized dextran with about 50% oxidation conversion (i.e., about half of the glucose rings in the dextran polymer are oxidized to dialdehydes) is prepared from dextran having a weight-average molecular weight of 8,500 to 11,500 Daltons (Sigma) by the method described by Cohen et al. (copending and commonly owned Patent Application No. PCT/US08/05013). A typical procedure is described here.

A 20-L reactor equipped with a mechanical stirrer, addition funnel, internal temperature probe, and nitrogen purge is charged with 1000 g of the dextran and 9.00 L of de-ionized water. The mixture is stirred at ambient temperature to dissolve the dextran and then cooled to 10 to 15° C. To the cooled dextran solution is added over a period of an hour, while keeping the reaction temperature below 25° C., a solution of 1000 g of sodium periodate dissolved in 9.00 L of de-ionized water. Once all the sodium periodate solution has been added, the mixture is stirred at 20 to 25° C. for 4 more hours. The reaction mixture is then cooled to 0° C. and filtered to clarify. Calcium chloride (500 g) is added to the filtrate, and the mixture is stirred at ambient temperature for 30 min and then filtered. Potassium iodide (400 g) is added to the filtrate, and the mixture is stirred at ambient temperature for 30 min. A 3-L portion of the resulting red solution is added to 9.0 L of acetone over a period of 10 to 15 min with vigorous stirring by a mechanical stirrer during the addition. After a few more minutes of stirring, the agglomerated product is separated from the supernatant liquid. The remaining red solution obtained by addition of potassium iodide to the second filtrate is treated in the same manner as above. The combined agglomerated product is broken up into pieces, combined with 2 L of methanol in a large stainless steel blender, and blended until the solid becomes granular. The granular solid is recovered by filtration and dried under vacuum with a nitrogen purge. The granular solid is then hammer milled to a fine powder. A 20-L reactor is charged with 10.8 L of de-ionized water and 7.2 L of methanol, and the mixture is cooled to 0° C. The granular solid formed by the previous step is added to the reactor and the slurry is stirred vigorously for one hour. Stirring is discontinued, and the solid is allowed to settle to the bottom of the reactor. The supernatant liquid is decanted by vacuum, 15 L of methanol is added to the reactor, and the slurry is stirred for 30 to 45 min while cooling to 0° C. The slurry is filtered in portions, and the recovered solids are washed with methanol, combined, and dried under vacuum with a nitrogen purge to give about 600 g of the oxidized dextran, which is referred to herein as D10-50.

The degree of oxidation of the product is determined by proton NMR to be about 50% (equivalent weight per aldehyde group=146). In the NMR method, the integrals for two ranges of peaks are determined, specifically, —O₂CHx- at about 6.2 parts per million (ppm) to about 4.15 ppm (minus the HOD peak) and —OCHx- at about 4.15 ppm to about 2.8 ppm (minus any methanol peak if present). The calculation of oxidation level is based on the calculated ratio (R) for these areas, specifically, R═(OCH)/(O₂CH).

### Preparation of Dextran Aldehyde (D60-20):

An oxidized dextran with about 20% oxidation conversion is prepared from dextran having a weight-average molecular weight of 60,000-90,000 Daltons (Sigma) using the procedure described above for D10-50. The concentration of the periodate used is adjusted to obtain the desired oxidation conversion of 20%.

### Preparation of Eight-Arm PEG 10K Octaamine (P8-10-1):

Eight-arm PEG 10K octaamine (Mₙ=10 kDa) is synthesized using the two-step procedure described by Chenault in co-pending and commonly owned U.S. Patent Application Publication No. 2007/0249870. In the first step, the 8-arm PEG 10K chloride is made by reaction of thionyl chloride with the 8-arm PEG 10K octaalcohol. In the second step, the 8-arm PEG 10K chloride is reacted with aqueous ammonia to yield the 8-arm PEG 10K octaamine. A typical procedure is described here.

The 8-arm PEG 10K octaalcohol (Mₙ=10000; NOF SunBright HGEO-10000), (100 g in a 500-mL round-bottom flask) is dried either by heating with stirring at 85° C. under vacuum (0.06 mm of mercury (8.0 Pa)) for 4 h or by azeotropic distillation with 50 g of toluene under reduced pressure (2 kPa) with a pot temperature of 60° C. The 8-arm PEG 10K octaalcohol is allowed to cool to room temperature and thionyl chloride (35 mL, 0.48 mol) is added to the flask, which is equipped with a reflux condenser, and the mixture is heated at 85° C. with stirring under a blanket of nitrogen for 24 hours. Excess thionyl chloride is removed by rotary evaporation (bath temp 40° C.). Two successive 50-mL portions of toluene are added and evaporated under reduced pressure (2 kPa, bath temperature 60° C.) to complete the removal of thionyl chloride. Proton NMR results from one synthesis are:

¹H NMR (500 MHz, DMSO-d6) δ 3.71-3.69 (m, 16H), 3.67-3.65 (m, 16H), 3.50 (s, ˜800H).

The 8-arm PEG 10K octachloride (100 g) is dissolved in 640 mL of concentrated aqueous ammonia (28 wt %) and heated in a pressure vessel at 60° C. for 48 hours. The solution is sparged for 1-2 hours with dry nitrogen to drive off 50 to 70 g of ammonia. The solution is then passed through a column (500 mL bed volume) of strongly basic anion exchange resin (Purolite® A-860, The Purolite Co., Bala-Cynwyd, Pa.) in the hydroxide form. The eluant is collected and three 250-mL portions of de-ionized water are passed through the column and also collected. The aqueous solutions are combined, concentrated under reduced pressure (2 kPa, bath temperature 60° C.) to about 200 g, frozen in portions and lyophilized to give the 8-arm PEG 10K octaamine, referred to herein as P8-10-1, as a colorless waxy solid.

### Preparation of Four-Arm PEG 2K Tetraamine (P4-2-1):

A 4-arm PEG 2K (Mₙ=2 kDa) tetraamine is prepared using a similar procedure as described above for the 8-arm PEG 10K octaamine. A typical procedure is described here.

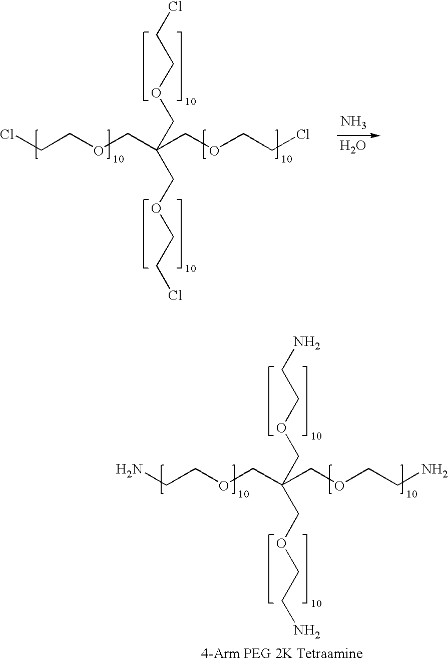

Four-arm PEG 2K tetraalcohol (Mₙ=2000; NOF SunBright PTE-2000), (100 g in a 500-mL round-bottom flask) is dissolved in 100 mL of dichloromethane. Thionyl chloride (88 mL, 1.2 mol) is added, and the mixture is stirred under a blanket of nitrogen at ambient temperature for 24 hours Excess thionyl chloride and dichloromethane are removed by rotary evaporation (bath temp 40° C.). Two successive 50-mL portions of toluene are added and evaporated under reduced pressure (2 kPa, bath temperature 60° C.) to complete the removal of thionyl chloride.

Proton NMR Results from One Preparation are:

¹H NMR (500 MHz, DMSO-d6): δ 3.71-3.68 (m, 8H), 3.67-3.65 (m, 8H), 3.57-3.55 (m, 8H), 3.50 (m, ˜140H), 3.47-3.45 (m, 8H), 3.31 (s, 8H).

The 4-arm PEG 2K tetrachloride (40 g) is dissolved in 600 mL of concentrated aqueous ammonia (28 wt %) and heated in a pressure vessel at 60° C. for 48 hours. The solution is cooled and sparged for 1.5 hours with dry nitrogen, and then concentrated by rotary evaporation (2 kPa, bath temperature 60° C.) to about 500 g. The solution is then passed through a column (500 mL bed volume) of strongly basic anion exchange resin (Purolite® A-860) in the hydroxide form. The eluant is collected, and two 250-mL portions of de-ionized water are passed through the column and collected. The aqueous fractions are combined and evaporated under reduced pressure (2 kPa, bath temperature 60° C.) to give the 4-arm PEG 2K tetraamine, referred to herein as P4-2-1, as a clear, pale-yellow liquid.

Proton NMR Results from One Preparation are:

¹H NMR (500 MHz, CDCl₃): δ 3.65-3.51 (m, ˜170H), 3.47 (m, 8H), 3.36 (s, 8H), 2.86 (t, J=5.3 Hz, 7.4H), 2.76 (t, J=5.4 Hz, 0.6H).

### General Methods

### Preparation of Hydrogel Precursor Solutions

Oxidized dextran solutions and multi-arm polyether amine solutions were prepared by dissolving the desired amount of oxidized dextran or multi-arm polyether amine in distilled water to achieve the desired concentration (wt %). The amine typically dissolved readily at room temperature. The oxidized dextran typically dissolved slowly at room temperature, but dissolved completely after heating at 37° C. overnight.

Various polyol additives were added to either the oxidized dextran or polyether amine solutions, or both. A formulation with an additive was designed by removing a quantity of water from a control formulation and replacing it with the same quantity of the additive.

### In Vitro Degradation Time Measurements

The degradation behavior of hydrogels at 37° C. in Dulbecco's phosphate buffered saline at pH 7.4 (DPBS, 1× without calcium or magnesium, Invitrogen, Carlsbad, Calif.; cat. 14190 or Mediatech, Herndon, Va.; cat. 21-031) was studied as follows. A double-barrel syringe (1:1, v/v) with a 16-step static mixing tip (Medmix Systems AG; Rotkreuz, Switzerland) was used to prepare a hydrogel test strip. The oxidized dextran solution was added to one side of the double-barrel syringe, and the polyether-amine solution was added to the other side. The mixing tip was cut 5 mm from the end to make a larger exit diameter.

A hydrogel formulation was cast using the double-barrel syringe with mixing tip into a 1 mm thick by 6.8 mm wide by approx. 70 mm long mold. After 15 minutes, the ends were trimmed and the resulting hydrogel strip was cut into 2 test strips, each 30 mm×6.8 mm×1 mm in size. After weighing, the strips were each placed in a 20 mL vial containing DPBS buffer. The vials were capped and placed in an incubator shaker at 37° C. and 80 rpm.

The hydrogel test strips were typically weighed at 2 hours and 5 hours on the first day, and every 24 hours thereafter until the weight of the test strip was less than 50% of its initial weight. At each time, the gel strip was removed from buffer, drained of excess liquid, and weighed. The strip was then placed in a vial with fresh DPBS and returned to the incubator.

This procedure resulted in a plot of gel weight versus time, expressed as % of initial weight versus time. Typically, there was an initial increase in weight due to equilibrium swelling, followed by some additional swelling as crosslinks are broken and finally a loss of weight as soluble degradation products diffuse from the gel. Fragments of the gel may linger for some time. The time to 50% of the initial weight was used as a meaningful parameter of the degradation curve for comparing formulations. This time, referred to herein as the degradation time, was estimated by interpolation between the time point at which the weight was just above 50% and the time point at which the weight was just below 50%. Reported values are averages of determinations on the two gel strip samples.

### Sag Distance Measurements

This method was designed to mimic the application of material on living tissue and the subsequent flow of the material on an inclined substrate. A heated plate at 37° C. was positioned at a fixed incline of 30 degrees. A copper plate was fabricated to fit on the heated surface. A fixed amount of formulation, 0.25 mL, was deposited near the top of the plate from a double-barrel syringe fitted with a 16-stage mixing tip, as described above, and a dispensing gun. The material flowed and then stopped. The distance of flow was determined and the average of 3 measurements was reported as the sag distance.

### Examples 1-4

### Effect of Poloxamer 407 on In Vitro Degradation Time of Hydrogels

The effect of Poloxamer 407 block copolymer addition on the degradation time of hydrogels was studied using a base formulation of 10 wt % D60-20 oxidized dextran in aqueous solution and 15 wt % P8-10-1 multi-arm PEG amine in aqueous solution. Formulations were prepared incorporating 10, 20 or 30% Poloxamer 407 in place of water in the PEG amine solution. The degradation times were measured as described in the General Methods section. The formulations and degradation times are shown in Table 1.

TABLE 1
Degradation Time of Hydrogels Containing Poloxamer 407

| | Oxidized | Multi-arm | | Degradation |
|---|---|---|---|---|
| | Dextran | PEG amine | Additive to PEG | Time |
| Example | Solution | Solution | amine Solution | (hours) |
| 1 | D60-20 | P8-10-1 | none | 3 |
| Comparative | 10 wt % | 15 wt % | | |
| 2 | D60-20 | P8-10-1 | Poloxamer 407 | 20 |
| | 10 wt % | 15 wt % | 10 wt % | |
| 3 | D60-20 | P8-10-1 | Poloxamer 407 | 60 |
| | 10 wt % | 15 wt % | 20 wt % | |
| 4 | D60-20 | P8-10-1 | Poloxamer 407 | >120 |
| | 10 wt % | 15 wt % | 30 wt % | |

As can be seen from the results in Table 1, the addition of Poloxamer 407 dramatically retards degradation of the hydrogels compared with the same formulation without the Poloxamer (Comparative Example 1). Poloxamer 407 by itself forms a gel in concentrated aqueous solutions at room temperature or above, but these gels degrade quickly. A 30% solution of Poloxamer 407 in water was prepared below room temperature and formed a gel when warmed to room temperature. A strip of this gel was placed in DPBS buffer at 37° C. and its degradation behavior was measured by the procedure described in the General Methods section. This gel had a degradation time of less than 2 hours. Examples 2, 3, and 4, with a combination of oxidized dextran, multi-arm PEG amine, and Poloxamer 407, had a much longer degradation time than either Poloxamer 407 alone or oxidized dextran and multi-arm PEG amine alone. Thus, combining all three of these components had a synergistic effect on degradation time.

### Examples 5-14

### Effect of Poloxamer and Poly(ethylene glycol) on In Vitro Degradation Time and Sag Distance of Hydrogels

Two types of Poloxamer block copolymers and two types of linear PEGs with alcohol end groups were evaluated as additives to oxidized dextran/multi-arm PEG amine hydrogels. The degradation times and sag distances were measured using the methods described in General Methods. The formulations, degradation and sag properties are shown in Table 2.

As can be seen from the results in Table 2, all of the additives retarded degradation compared to the controls without an additive (Examples 5 and 10, Comparative). In several cases the retardation was dramatic. The additives also reduced sag distance, which is desirable because excessive flow of material upon application of the components to a tissue site is undesirable.

TABLE 2
Degradation Time and Sag Distance of Hydrogels Containing
Polyol Additives

| | | Multi-arm | Additive to | De- | Sag |
|---|---|---|---|---|---|
| | Oxidized | PEG | PEG | gradation | Dis- |
| | Dextran | amine | amine | Time | tance |
| Example | Solution | Solution | Solution | (hours) | (cm) |
| 5 | D60-20 | P8-10-1 | none | 2 | 19 |
| Comparative | 10 wt % | 15 wt % | | | |
| 6 | D60-20 | P8-10-1 | Poloxamer | 5 | 12 |
| | 10 wt % | 15 wt % | 188 | | |
| | | | 10 wt % | | |
| 7 | D60-20 | P8-10-1 | Poloxamer | 21 | 9 |
| | 10 wt % | 15 wt % | 407 | | |
| | | | 10 wt % | | |
| 8 | D60-20 | P8-10-1 | Linear | 5 | 11 |
| | 10 wt % | 15 wt % | PEG 8k | | |
| | | | 10 wt % | | |
| 9 | D60-20 | P8-10-1 | Linear | 8 | 12 |
| | 10 wt % | 15 wt % | PEG 14k | | |
| | | | 10 wt % | | |
| 10 | D10-50 | P8-10-1 | none | 7 | >20 |
| Comparative | 6 wt % | 18 wt % | | | |
| 11 | D10-50 | P8-10-1 | Poloxamer | 58 | 15 |
| | 6 wt % | 18 wt % | 188 | | |
| | | | 10 wt % | | |
| 12 | D10-50 | P8-10-1 | Poloxamer | 69 | 13 |
| | 6 wt % | 18 wt % | 407 | | |
| | | | 10 wt % | | |
| 13 | D10-50 | P8-10-1 | Linear | 64 | 14 |
| | 6 wt % | 18 wt % | PEG 8k | | |
| | | | 10 wt % | | |
| 14 | D10-50 | P8-10-1 | Linear | 80 | 13 |
| | 6 wt % | 18 wt % | PEG 14k | | |
| | | | 10 wt % | | |

### Examples 15-39

### Effect of Poly(ethylene glycol) Addition to a Range of Hydrogel Formulations on In Vitro Degradation Time and Sag Distance

To demonstrate the effect of adding poly(ethylene glycol) of 14,000 number-average molecular weight (linear PEG 14k) to a range of hydrogel formulations, a statistically meaningful set of 25 formulations was designed based on the components oxidized dextran D10-50, multi-arm PEG amine P8-10-1, linear PEG 14k, and water. Degradation times and sag distances were determined for each formulation using the methods described in General Methods. The formulations, degradation and sag properties are shown in Table 3.

One way to evaluate these results is to calculate the average degradation time and the average sag distance for formulations at each level of linear PEG 14k. These averages are shown in Table 4. As can be seen from the data in Table 4, linear PEG 14k addition retards degradation and reduces sag distance for oxidized dextran/multi-arm PEG-amine hydrogel formulations.

For this set of formulations, degradation time varied from 1 to 215 hours, and sag distance varied from 5 to 20 cm. Thus, only simple adjustments in the formulation are needed to achieve a broad range of properties that are critical to performance.

TABLE 3
Degradation Time and Sag Distance of Hydrogels Containing
Linear PEG 14k

| | | | Linear PEG | | |
|---|---|---|---|---|---|
| | | | 14k | De- | |
| | | | (wt % in PEG | gradation | Sag |
| | D10-50 | P8-10-1 | amine | Time | Distance |
| Example | (wt %) | (wt %) | solution) | (hours) | (cm) |
| 15 | 5 | 18 | 14 | 21 | 16 |
| 16 | 5 | 18 | 7 | 4 | 19 |
| 17 | 5 | 30 | 0 | 1 | 14 |
| Comparative | | | | | |
| 18 | 6.5 | 30 | 14 | 54 | 9 |
| 19 | 6.5 | 30 | 0 | 5 | 9 |
| Comparative | | | | | |
| 20 | 8 | 30 | 14 | 69 | 7 |
| 21 | 8 | 18 | 0 | 20 | >20 |
| Comparative | | | | | |
| 22 | 5 | 24 | 14 | 12 | 11 |
| 23 | 8 | 24 | 7 | 61 | 11 |
| 24 | 8 | 24 | 14 | 178 | 9 |
| 25 | 6.5 | 18 | 14 | 52 | 13 |
| 26 | 8 | 18 | 7 | 59 | 12 |
| 27 | 8 | 18 | 0 | 15 | 17 |
| Comparative | | | | | |
| 28 | 5 | 30 | 14 | 14 | 11 |
| 29 | 8 | 18 | 14 | 215 | 10 |
| 30 | 6.5 | 24 | 7 | 15 | 12 |
| 31 | 5 | 18 | 0 | 4 | >20 |
| Comparative | | | | | |
| 32 | 5 | 24 | 0 | 1 | 18 |
| Comparative | | | | | |
| 33 | 5 | 30 | 7 | 4 | 11 |
| 34 | 8 | 24 | 0 | 24 | 11 |
| Comparative | | | | | |
| 35 | 8 | 30 | 14 | 63 | 5 |
| 36 | 8 | 30 | 7 | 58 | 7 |
| 37 | 5 | 18 | 0 | 4 | >20 |
| Comparative | | | | | |
| 38 | 8 | 30 | 0 | 20 | 7 |
| Comparative | | | | | |
| 39 | 5 | 24 | 7 | 5 | 16 |

TABLE 4
Average Degradation Times and Sag Distances

| Linear PEG 14k | Average Degradation | Average Sag Distance |
|---|---|---|
| (wt %) | Time (hours) | (cm) |
| 0 | 10.4 | 15.4 |
| 7 | 29.6 | 12.6 |
| 14 | 75.3 | 10.2 |

### Examples 40-41

### Effect of the Manner of Addition of Branched Polyethylene glycol) to Hydrogel Formulation on In Vitro Degradation Time

An 8-arm poly(ethylene glycol), P8-10-1-OH, was evaluated as an additive to the hydrogel formed by mixing an 8 wt % D10-50 oxidized dextran solution with a 30 wt % P8-10-1 multi-arm PEG amine solution. In one hydrogel formulation (Example 40), 7 wt % P8-10-1-OH was added to the oxidized dextran solution. In a second hydrogel formulation (Example 41), 7 wt % P8-10-1-OH was added to the multi-arm PEG amine solution. The in vitro degradation time was determined for these two hydrogel formulations as described above. The first formulation (Example 40) had a degradation time of 79 hours. The second formulation (Example 41) had a degradation time of 47 hours. Therefore, the degradation time was significantly longer when the P8-10-1-OH additive was added to the oxidized dextran solution than when it was added to the multi-arm PEG amine solution.

### Examples 42-44

### Effect of Poly(ethylene glycol) on In Vitro Degradation Time of Hydrogels

A linear poly(ethylene glycol) of 4,600 number-average molecular weight, linear PEG 4.6 k, and an 8-arm poly(ethylene glycol), P8-10-1-OH, were evaluated as additives to the hydrogel formed by mixing an 8 wt % D10-50 oxidized dextran solution with a 30 wt % P8-10-1 multi-arm PEG amine solution. The hydrogel formulation without any additives was also prepared (Example 42, Comparative). In a second hydrogel formulation (Example 43), 10 wt % Linear PEG 4.6 k was added to the oxidized dextran solution. In a third hydrogel formulation (Example 44), 10 wt % P8-10-1-OH was added to the oxidized dextran solution.

The degradation times determined for these three hydrogel formulations were 37, 89, and 114 hours for Examples 42, 43, and 44, respectively. Therefore, both poly(ethylene glycol) additives retarded the degradation of the hydrogel significantly compared to the hydrogel formulation without an additive (Example 42, Comparative).

### Examples 45-47

### Effect of Poly(vinyl alcohol) on In Vitro Degradation Time of Hydrogels

Poly(vinyl alcohol) of weight-average molecular weight 13,000 to 23,000, either 88% (PVOH 88) or 98% (PVOH 98) hydrolyzed, was evaluated as an additive to the hydrogel formed by mixing an 8 wt % D10-50 oxidized dextran solution with a 30 wt % P8-10-1 multi-arm PEG amine solution. The hydrogel formulation without any additives was also prepared (Example 45, Comparative). In a second hydrogel formulation (Example 46), 10 wt % PVOH 88 was added to the oxidized dextran solution. In a third hydrogel formulation (Example 47), 10 wt % PVOH 98 was added to the oxidized dextran solution.

The degradation times determined for these three hydrogel formulations were 30, 88, and 102 hours for Examples 45, 46, and 47, respectively. Therefore, both poly(vinyl alcohol) additives retarded the degradation of the hydrogel significantly compared to the hydrogel without an additive (Example 45, Comparative).

### Example 48

### Effect of Random Copolymer of Ethylene Glycol and Propylene Glycol on In Vitro Degradation Time of Hydrogel

A random copolymer of ethylene glycol and propylene glycol, poly(EG-ran-PG), was evaluated as an additive to the hydrogel formed by mixing an 8 wt % D10-50 oxidized dextran solution with a 30 wt % P8-10-1 multi-arm PEG amine solution. The 10 wt % poly(EG-ran-PG) was added to the oxidized dextran solution. The degradation time for this hydrogel formulation was determined to be 66 hours, as compared to 30 hours for the same formulation without the additive (Example 45, Comparative). Therefore, the additive poly(EG-ran-PG) retarded the degradation of the hydrogel significantly.

### Examples 49 and 50

### Effect of Linear Polyethylene Glycol Having an Average Molecular Weight of 35,000 Daltons on In Vitro Degradation Time of Hydrogels

A linear PEG alcohol having a number-average molecular weight of about 35,000 Daltons (Mₙ=35k; Sigma-Aldrich, cat. 81310) was evaluated as an additive to the hydrogel formed by mixing a solution containing P8-10-1 multi-arm PEG amine and P4-2-1 multi-arm PEG amine with a solution containing D10-49 oxidized dextran. The P8-10-1 multi-arm PEG amine, the P4-2-1 multi-arm PEG amine, and the linear PEG alcohol were added to water in a weight ratio of 27:3:5 to provide an aqueous solution containing 35 wt % total solids. The resulting PEG solution was mixed in a 1:1 volume ratio with an aqueous solution containing 10 wt % D10-49 oxidized dextran using a double-barrel syringe equipped with a static mixing tip to form hydrogel strips. The degradation time of the hydrogel strips was determined as described in General Methods, except that the degradation time was recorded as the time for the hydrogels to completely degrade.

For comparison, hydrogel strips were prepared without the addition of the linear PEG alcohol. Specifically, the P8-10-1 multi-arm PEG amine and the P4-2-1 multi-arm PEG amine were added to water in the weight ratio of 27:3 to provide an aqueous solution containing 30 wt % total solids. The resulting PEG solution was mixed in a 1:1 volume ratio with an aqueous solution containing 10 wt % D10-49 oxidized dextran using a double-barrel syringe equipped with a static mixing tip, as described above.

The degradation time of the hydrogels is given in Table 5. As can be seen from the results, the linear PEG alcohol having a number-average molecular weight of about 35,000 Daltons resulted in a significant increase in the degradation time of the hydrogel compared to the hydrogel without the additive.

TABLE 5
Degadation Time of Hydrogels Containing
Linear 35k PEG Alcohol

| | | Time for Complete |
|---|---|---|
| Example | Additive | Degradation (hours) |
| 49 | linear 35k PEG alcohol | 72 |
| 50, Comparative | none | 48 |

### Examples 51-53

### Effect of Dextrans Having Different Molecular Weights on In Vitro Degradation Time of Hydrogels

Dextrans having average molecular weights of 450,000 Da (dextran 450k; Sigma, cat. D1037) and 2,000,000 Da (dextan 2,000k; Sigma, cat. D5376) were evaluated as an additive to the hydrogel formed by mixing a solution containing P8-10-1 multi-arm PEG amine and a P4-2-1 multi-arm PEG amine with a solution containing D10-49 oxidized dextran. The P8-10-1 multi-arm PEG amine and the P4-2-1 multi-arm PEG amine were added to water in a weight ratio of 27:3 to provide an aqueous solution containing 30 wt % total solids. The D10-49 oxidized dextran and the dextran additive, as shown in Table 6, were added to water in a weight ratio of 10:1 to form an aqueous solution containing 11 wt % total solids. The PEG solution was mixed in a 1:1 volume ratio with a dextran solution using a double-barrel syringe equipped with a static mixing tip to form hydrogel strips. The degradation time of the hydrogel strips was determined as described in General Methods, except that the degradation time was recorded as the time for the hydrogels to completely degrade.

For comparison, hydrogel strips were prepared without the addition of the dextran additive. Specifically, the P8-10-1 multi-arm PEG amine and the P4-2-1 multi-arm PEG amine were added to water in the weight ratio of 27:3 to provide an aqueous solution containing 30 wt % total solids. The resulting PEG solution was mixed in a 1:1 volume ratio with an aqueous solution containing 10 wt % D10-49 oxidized dextran using a double-barrel syringe equipped with a static mixing tip, as described above.

The degradation time of the hydrogels is given in Table 6. As can be seen from the results, the dextran additives resulted in a significant increase in the degradation time of the hydrogel compared to the hydrogel without the additive.

TABLE 6
Degadation Time of Hydrogels Containing Dextran

| | | Time for Complete |
|---|---|---|
| Example | Additive | Degradation (hours) |
| 51 | Dextran 450k | 144 |
| 52 | Dextran 2,000k | 144 |
| 53, Comparative | none | 48 |

What is claimed is:

1. A kit comprising:
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
provided that:
(i) the water-dispersible polyol does not induce gelation when mixed in an aqueous medium with either (a) alone or (b) alone; and
(ii) if said water-dispersible polyol is a polysaccharide, then said polysaccharide has a weight-average molecular weight greater than 5,000 to about 2,000,000 Daltons.

2. The kit according to claim 1 wherein the oxidized polysaccharide is contained in a first aqueous solution or dispersion, the water-dispersible multi-arm amine is contained in a second aqueous solution or dispersion, and the water-dispersible polyol is contained in at least one of: (i) the first aqueous solution or dispersion; (ii) the second aqueous solution or dispersion; or (iii) a third aqueous solution or dispersion.

3. The kit according to claim 2 wherein the first aqueous solution or dispersion comprises the oxidized polysaccharide at a concentration of about 5% to about 40% by weight relative to the total weight of the solution or dispersion.

4. The kit according to claim 2 wherein the second aqueous solution or dispersion comprises the water-dispersible multi-arm amine at a concentration of about 5% to about 70% by weight relative to the total weight of the solution or dispersion.

5. The kit according to claim 2 wherein at least one of the first aqueous solution or dispersion, the second aqueous solution or dispersion, or the third aqueous solution or dispersion comprises the water-dispersible polyol at a concentration of about 1% to about 30% by weight relative to the total weight of the solution or dispersion.

6. The kit according to claim 1 wherein the oxidized polysaccharide is selected from the group consisting of oxidized derivatives of: dextran, carboxymethyldextran, starch, agar, cellulose, hydroxyethylcellulose, carboxymethylcellulose, pullulan, inulun, levan, and hyaluronic acid.

7. The kit according to claim 6 wherein the oxidized polysaccharide is oxidized dextran.

8. The kit according to claim 1 wherein the water-dispersible, multi-arm amine is selected from the group consisting of water dispersible multi-arm polyether amines, amino-terminated dendritic polyamidoamines, and multi-arm branched end amines.

9. The kit according to claim 8 wherein the water dispersible multi-arm polyether amines are selected from the group consisting of amino-terminated star polyethylene oxides, amino-terminated dendritic polyethylene oxides, amino-terminated comb polyethylene oxides, amino-terminated star polypropylene oxides, amino-terminated dendritic polypropylene oxides, amino-terminated comb polypropylene oxides, amino-terminated star polyethylene oxide-polypropylene oxide copolymers, amino-terminated dendritic polyethylene oxide-polypropylene oxide copolymers, amino-terminated comb polyethylene oxide-polypropylene oxide copolymers, and polyoxyalkylene triamines.

10. The kit according to claim 1 wherein the water-dispersible polyol is selected from the group consisting of linear or branched poly(ethylene glycol), block or random copolymers of ethylene glycol and propylene glycol, poly(vinyl alcohol), and polysaccharides.

11. The kit according to claim 1 wherein the oxidized polysaccharide, the water-dispersible, multi-arm amine, and the water-dispersible polyol are in the form of finely divided powders.

12. A dried hydrogel formed by a process comprising the steps of:
a) combining in a solvent (i) at least one oxidized polysaccharide containing aldehyde groups, said oxidized polysaccharide having a weight-average molecular weight of about 1,000 to about 1,000,000 Daltons and having an equivalent weight per aldehyde group of about 65 to about 1500 Daltons with (ii) at least one water-dispersible, multi-arm polyether amine having at least three arms terminated by at least one primary amine group, said multi-arm polyether amine having a number-average molecular weight of about 450 to about 200,000 Daltons, and (iii) an effective amount of at least one water-dispersible polyol having a molecular weight greater than 2,000 to about 2,000,000 Daltons, said polyol containing two or more hydroxyl groups per molecule, to form a hydrogel, and
b) treating said hydrogel to remove at least a portion of said solvent to form the dried hydrogel;
provided that:
(A) the water-dispersible polyol does not induce gelation when mixed in an aqueous medium with either (i) alone or (ii) alone; and
(B) if said water-dispersible polyol is a polysaccharide, then said polysaccharide has a weight-average molecular weight greater than 5,000 to about 2,000,000 Daltons.

13. The dried hydrogel according to claim 12 wherein said dried hydrogel is in the form of a film.

14. The dried hydrogel according to claim 12 wherein the process further comprises the step of comminuting the dried hydrogel to form finely divided particles.

15. A composition comprising the reaction product of:
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
provided that:
(i) the water-dispersible polyol does not induce gelation when mixed in an aqueous medium with either (a) alone or (b) alone; and
(ii) if said water-dispersible polyol is a polysaccharide, then said polysaccharide has a weight-average molecular weight greater than 5,000 to about 2,000,000 Daltons.

16. A method for applying a coating to an anatomical site on tissue of a living organism comprising:
applying to the site
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
provided that:
(i) the water-dispersible polyol does not induce gelation when mixed in an aqueous medium with either (a) alone or (b) alone; and
(ii) if said water-dispersible polyol is a polysaccharide, then said polysaccharide has a weight-average molecular weight greater than 5,000 to about 2,000,000 Daltons;
wherein (a), (b), and (c) are applied to the site in any order, or (a), (b), and (c) are premixed and the resulting mixture is applied to the site before the mixture completely cures.

17. A method for bonding at least two anatomical sites together comprising:
applying to at least one of the at least two anatomical sites:
a) at least one oxidized polysaccharide containing aldehyde groups, having a weight-average molecular weight of about 1,000 to about 1,000,000 Daltons, said oxidized polysaccharide having an equivalent weight per aldehyde group of about 65 to about 1500 Daltons;
b) at least one water-dispersible, multi-arm amine having at least three arms terminated by at least one primary amine group, wherein the multi-arm amine has a number-average molecular weight of about 450 to about 200,000 Daltons; and
c) an effective amount of at least one water-dispersible polyol having a weight-average molecular weight greater than 2,000 to about 2,000,000 Daltons, said polyol containing two or more hydroxyl groups per molecule;
provided that:
(i) the water-dispersible polyol does not induce gelation when mixed in an aqueous medium with either (a) alone or (b) alone; and
(ii) if said water-dispersible polyol is a polysaccharide, then said polysaccharide has a weight-average molecular weight greater than 5,000 to about 2,000,000 Daltons;
or premixing (a), (b) and (c) and applying the resulting mixture to at least one site before the resulting mixture completely cures; and
contacting the at least two anatomical sites together.

18. A method for increasing degradation time of a hydrogel formed from at least one oxidized polysaccharide (component A) and at least one water-dispersible, multi-arm amine (component B), said at least one oxidized polysaccharide containing aldehyde groups, having a weight-average molecular weight of about 1,000 to about 1,000,000 Daltons and an equivalent weight per aldehyde group of about 65 to about 1500 Daltons, and said at least one water-dispersible, multi-arm amine having at least three arms terminated by at least one primary amine group, and a number-average molecular weight of about 450 to about 200,000 Daltons; said method comprising:
contacting component A and component B in the presence of an aqueous medium and at least one water-dispersible polyol having a weight-average molecular weight greater than 2,000 to about 2,000,000 Daltons, said polyol containing two or more hydroxyl groups per molecule to form a mixture that forms a resulting hydrogel, wherein, in said method, the water-dispersible polyol is used in an amount sufficient to increase the degradation time of the resulting hydrogel under predetermined conditions by at least about 10% compared to that of the hydrogel formed under said conditions, but in the absence of said water-dispersible polyol;
provided that:
(i) the water-dispersible polyol does not induce gelation when mixed in an aqueous medium with either component A alone or component B alone; and
(ii) if said water-dispersible polyol is a polysaccharide, then said polysaccharide has a weight-average molecular weight greater than 5,000 to about 2,000,000 Daltons.
